(19) United States
(12) Patent Application Publication
(10) Pub. No.: US 2005/0064585 A1
(43) Pub. Date: Mar. 24, 2005

(54) Viral concentration process

(76) Inventors: William Wolf, Woollahra (AU); Mark Angles, Vaucluse (AU); Monica Logan, Castle Hill (AU)

Correspondence Address: CHRISTIE, PARKER & HALE, LLP, PO BOX 7068, PASADENA, CA, 91109-7068, US

(21) Appl. No.: 10/667,076

(22) Filed: Sep. 18, 2003

(51) Int. Cl. C12M 1/12

(52) U.S. Cl. 435/297.4; 435/308.1

(57) ABSTRACT

An apparatus for isolating a microorganism from a liquid, the apparatus comprising: a first endcap engageable with an inlet end of a hollow fibre filter, the first endcap including a first passage having an inlet engageable with a liquid input conduit; and an outlet into the filter; and a second endcap engageable with an outlet end of the hollow fibre filter, the second endcap including a second passage having an outlet engageable with a liquid return conduit, and an inlet from the filter; the first passage and the second passage being independently sized such that in conjunction with a flow restriction means which restricts a flow of the liquid through the second passage, a predetermined exit liquid flow rate from at least one permeate outlet of the filter is achieved; the microorganism is captured within the hollow fibre filter; and the maximum working pressure of the hollow fibre filter is not exceeded.

### FIELD OF THE INVENTION

The present invention relates to the field of detection and concentration of viruses from a sample of liquid.

### BACKGROUND OF THE INVENTION

The presence of human enteric viruses in an environmental water source have been shown to indicate the incidence of human faecal contamination of the water source. Public health issues, in particular in the event of pathogenic viruses in water sources with an end use for human consumption or use, are raised in respect of water source contamination. As a consequence of public health issues, water supply sources are tested and analysed by water regulation authorities for monitoring risks to the public and also for tracking inputs of human faecal matter into water catchments.

Due to the relatively low concentration in a water supply, and size of a virus, in the order of one millionth of a millimetre, it is necessary to concentrate large volumes of water using an ultrafitration technique in order to isolate viral particles. Typically, this is done by filtering a water sample using ultra filtration membranes or filters having pore sizes less than the size of a virus. The ultra filtration membrane or filter effectively traps the virus which is then recovered for analysis.

Several techniques and developments exist in the field of a viral concentration using ultra filtration techniques. Such concentration techniques used include the use of reusable filters including Amicon® microfiltration filters by Millipore. Using a reusable filter for the concentration and detection of a virus from a liquid sample includes the necessity of performing a decontamination step between samples, which is effectively equivalent to running another sample and includes added time and cost in processing. A negative control must also be run on each cleaned reusable unit to ensure that the risk of cross contamination between samples is minimised, also adding to the time and cost of processing. Furthermore, a positive control must be run with each batch of samples to determine the recovery efficiency of the process. To ensure that there is no cross contamination of samples by the positive control, reusable filters must be cleaned and the negative control run on the cleaned filter. A filter used to run a positive control must be placed in quarantine until the outcome of the negative control is determined. This effectively removes a number of filters from the work flow, while results on the negative controls are forthcoming. From an Occupational Health and Safety aspect, the use of live attenuated virus as positive controls introduces the risk of contamination of the work place or exposure of analysts to live virus. In addition, the use of sodium hypochlorite (bleach) to clean filtration units increased operator exposure to a hazardous chemical.

Juliano & Sobsey (1997) reported using a disposable dialysis filter (Primus 2000, Minntech Minneapolis Minn.), in order to concentrate bacteria, viruses and protozoa from water. Simmons et al. (2001), reported using a Hemoflow F80A cartridge to concentrate Cryptosporidium oocysts from a sample of water. Both of these methods describe a filtration system using 5 over 16th inch ID tubing and pressure gauges.

### SUMMARY OF THE INVENTION

In a first aspect, the present invention is an apparatus for isolating a microorganism from a liquid, the apparatus comprising:
a first endcap engageable with an inlet end of a hollow fibre filter, the first endcap including a first passage having an inlet engageable with a liquid input conduit; and an outlet into the filter; and
a second endcap engageable with an outlet end of the hollow fibre filter, the second endcap including a second passage having an outlet engageable with a liquid return conduit, and an inlet from the filter;
   the first passage and the second passage being independently sized such that in conjunction with a flow restriction means which restricts a flow of the liquid through the second passage, a predetermined exit liquid flow rate from at least one permeate outlet of the filter is achieved;
   the microorganism is captured within the hollow fibre filter; and
   the maximum working pressure of the hollow fibre filter is not exceeded.

In a first embodiment of the first aspect, the hollow fibre filter is a haemodialysis filter.

In a second embodiment of the first aspect the flow restriction means is selected from the group comprising a clamp means for compressing the liquid exit conduit, a flow restriction valve, a throttle valve and the inherent size of the second passage.

In an embodiment of the first aspect, the liquid is a finite liquid sample supply and the first endcap further comprises a pressure relief valve, wherein the pressure relief valve provides a fluid pathway from the inlet end of the filter to the finite liquid sample supply when the liquid pressure applied to the filter approaches the maximum working pressure of the filter such that the maximum working pressure of the filter is not exceeded. Preferably, the pressure relief valve is formed from a sterilisable material.

In another embodiment of the present aspect, the first endcap and the second endcap are formed of a sterilisable material. Preferably the sterilisable material is a metallic material. More preferably, the metallic material is stainless steel.

In a further embodiment of the first aspect, the hollow fibre filter is a haemodialysis filter. Preferably the predetermined exit liquid flow rate is in the range of from about 0.25 l/min to about 3.0 l/min, more preferably about 1.5 l/min, and the maximum working pressure of the hollow fibre filter is about 25 psi. Preferably the first passage and the second passage are circular, and have a diameter in the range from about 6 mm to about 30 mm. More preferably, the first passage and the second passage have a diameter of about 21 mm.

In a preferred embodiment of the first aspect the first endcap and the second endcap are engageable with the liquid input conduit and the liquid return conduit by an adaptor member, wherein an adaptor is affixed to the liquid input conduit and the liquid return conduit, and the adaptor member is releasably engageable with the endcaps by a clamp means. Preferably the adaptor member is affixed to the liquid input conduit and the liquid return conduit by a hoseclamp. Preferably the hoseclamps are formed from a sterilisable material, more preferably the sterilisable material is stainless steel.

Preferably the liquid input conduit, the first endcap, the filter, the second endcap and the liquid return conduit are connected in a loop arrangement, and the finite liquid sample supply is introduced into the loop arrangement between the liquid input conduit and the liquid return conduit. Preferably the liquid input conduit, the liquid return conduit and a feed conduit from the finite liquid sample supply are fluidly connected by a T-junction member. Preferably the total volume of the loop arrangement and the feed conduit is in the range of from about 250 ml to about 400 ml, more preferably the total volume of the loop arrangement and the feed conduit is about 340 ml.

Preferably the finite liquid sample supply is introduced into the closed loop arrangement by venturi effect. Preferably a filtration mesh is arranged between the finite liquid sample supply and the closed loop arrangement and the filtration mesh has a pore size of about 100 μm and a diameter of about 13 mm. Preferably the filtration mesh is formed from stainless steel.

Preferably the finite liquid sample supply has a volume in the range of from about 5 litres to about 25 litres, more preferably about 10 litres, still more preferably the finite liquid sample supply is contained within a FranRica™ bag (FMC Foodtech). Preferably the liquid input conduit, the liquid return conduit and the feed conduit are formed from a silicon-peroxide material.

In yet another embodiment of the first aspect, the liquid inlet conduit includes a portion engageable with a Peristaltic pump. Preferably the liquid is pumped through the closed loop arrangement by means of a Peristaltic pump applied to the liquid input conduit and restriction of liquid flow rate through the liquid return conduit is effected by a clamping means constricting the liquid return conduit.

In yet another embodiment of the first aspect, the microorganism is selected from the group of bacteria, protozoa or viruses. Preferably, the protozoa is selected from the group including Cryptosporidium and Giardia. Preferably, the virus is selected from those of the group including enterovirus, hepatitis A, rotavirus, noroviruses, astrovirus, reovirus, adenovirus and bacteriophage.

In a second aspect, the present invention is a method for isolating a microorganism from a liquid, the method comprising the step of:
(i) capturing and concentrating the microorganism on a hollow fibre filter by passing a sample of the liquid through the hollow fibre filter, the hollow fibre filter having a first endcap engaged with the inlet end of a hollow fibre filter and a second endcap engageable with the outlet end of the hollow fibre filter, the first endcap including a first passage having an inlet engageable with a liquid input conduit; and an outlet into the filter so as to provide a fluid pathway between the filter and a liquid input conduit, and the second endcap including a second passage having an outlet engageable with a liquid return conduit, and an inlet from the filter so as to provide a fluid pathway between the filter and a liquid return conduit;
   wherein the size of first passage and the second passage have been predetermined such that upon restriction of liquid flow rate through the liquid return conduit, a predetermined exit liquid flow rate from at least one permeate outlet of the hollow fibre filter is achieved; and
   the pressure of the liquid passed through the hollow fibre filter is less than the maximum working pressure of the hollow fibre filter.

In a first embodiment of the second aspect, the method further comprises a step of removing the captured microorganism from the hollow fibre filter. Preferably, the method further comprises a step of further concentrating the captured microorganism.

In a preferred embodiment of the second aspect, the hollow fibre filter is a haemodialysis filter. Preferably, the predetermined exit liquid flow rate is in the range of from about 0.5 l/min to about 3.0 l/min, more preferably about 1.5 l/min, and the maximum working pressure of the hollow fibre filter is about 25 psi. Preferably the first passage and the second passage first endcap and the second endcap are circular in shape and the first passage, and the second passage have a diameter in the range from about 6 mm to about 30 mm. More preferably the first passage and the second passage have a diameter of about 21 mm.

Preferably the liquid input conduit, the first endcap, the filter, the second endcap and the liquid return conduit form are connected in a loop arrangement, and the finite liquid sample supply is introduced into the loop arrangement between the liquid input conduit and the liquid return conduit via a feed conduit. Preferably the liquid input conduit, the liquid return conduit and the feed conduit from the finite liquid sample supply are fluidly connected by a T-junction member. Preferably the total volume of the loop arrangement and the feed conduit is in the range of from about 250 ml to about 400 ml, more preferably about 340 ml. Preferably the finite liquid sample supply is introduced into the closed loop arrangement by venturi effect and a filtration mesh is arranged between the finite liquid sample supply and the closed loop arrangement as a pre-filtering means. Preferably the filtration mesh has a pore size of about 100 μm and a diameter of about 13 mm.

Preferably the finite liquid sample supply has a volume in the range of from about 5 litres to about 25 litres, more preferably about 10 litres and still more preferably the finite liquid sample supply is contained within a FranRica™ bag. Air contained within the closed loop arrangement is purged from the closed loop arrangement. Preferably the air purged from the closed loop arrangement is returned from a pressure release valve located at the inlet side of the filter, to the FranRica™ bag from a pressure release valve located at the inlet side of the filter by means of a conduit.

Preferably the liquid is pumped through the closed loop arrangement by means of a Peristaltic pump applied to the liquid input conduit and restriction of liquid flow rate through the liquid return conduit is effected by a clamping means constricting the liquid return conduit. Preferably the finite liquid sample supply contains a non-ionic surface active agent, more preferably the non-ionic surface active agent is Polyoxyethylene Sorbitan Monooleate, for example Tween-80™, in a concentration of bout 0.005% v/v with the finite liquid sample supply.

In another embodiment of the second aspect, the step of capturing and concentrating the microorganism further comprises passing a buffer solution through the closed loop arrangement when about 50 ml to about 100 ml of residual liquid remains in the FranRica™ bag, wherein the liquid return conduit is unconstricted during passing of the buffer solution through the closed loop arrangement. The liquid input conduit, the liquid return conduit and feed conduit are disassociated; the remaining liquid in the finite liquid sample supply and the feed conduit are transferred to a container having a first predetermined volume of buffer solution; and the buffer solution is drawn from the container through the liquid inlet conduit, passed through the hollow fibre filter, passed through the liquid return conduit removed from the closed loop arrangement and returned to the container, until a second predetermined volume of buffer solution remains in the container; wherein the remaining liquid in the liquid inlet conduit, the hollow fibre filter and the liquid return conduit is transferred to the container; nitrogen gas is used to purge any remaining liquid from the loop arrangement; and the filtration mesh is transferred to the container. Preferably the first predetermined volume of buffer solution is about 600 ml and the second predetermined volume of buffer solution is about 300 ml. Preferably the buffer solution is a carbonate buffer solution and preferably has a pH value of about 9.6.

In another embodiment of the second aspect, the step of removing the captured microorganism from the hollow fibre filter comprises disengaging the first endcap and the second endcap from the hollow fibre filter; closing the at least one permeate outlet of the filter; and backwashing the hollow fibre filter with a third predetermined volume of buffer solution such that the third volume of buffer solution is transferred to the container; wherein the microorganism from the finite liquid sample supply is concentrated in the buffer solution in the container. Preferably the third predetermined volume of buffer solution is about 200 ml. Preferably the hollow fibre filter is backwashed using a syringe.

In yet another embodiment of the second aspect, the step of further concentrating the microorganism includes precipitation using polyethylene glycol-6000 concentration techniques. Preferably the type of microorganism present in the captured microorganism is determined by analysis and the concentration of the microorganism present in the finite liquid sample supply is determined.

In yet a further embodiment of the second aspect, the microorganism is selected from the group of bacteria, protozoa or viruses. Preferably, the protozoa is selected from the group including Cryptosporidium and Giardia. Preferably, the virus is selected from those of the group including enterovirus, hepatitis A, rotavirus, noroviruses, astrovirus, reovirus, adenovirus and bacteriophage.

In a third aspect, the present invention is an endcap for engagement with a hollow fibre filter, the endcap comprising:
a first end engageable with an end of the hollow fibre filter;
a passage having an inlet engageable with a liquid input conduit; and
an outlet into the filter;
   wherein the passage has a diameter in the range of from about 6 mm to about 30 mm.

In a preferred embodiment of the third aspect, the hollow fibre filter is a haemodialysis filter. Preferably the passage has a diameter of about 21 mm.

In another embodiment of the third aspect, the endcap further comprising a pressure relief valve, wherein the pressure relief valve provides a fluid pathway from the passage to atmosphere. Preferably the endcap and/or the pressure relief valve is formed from a sterilisable material, more preferably the sterilisable material is stainless steel.

In a fourth aspect, the present invention is a concentrated microorganism when concentrated according to the method of the second aspect.

In a fifth aspect the present invention is an apparatus for calibrating a pressure relief valve, the apparatus comprising:
an endcap according to the first aspect;
a pressure relief valve engaged with the endcap so as to provide a fluid pathway between the passage of the endcap and an exit port located on the pressure relief valve; and
a hydrostatic pump, the hydrostatic pump including a pressure indication means;
   wherein when a fluid is pumped through the endcap by the hydrostatic pump, the pressure indication means indicates the pressure at which the threshold pressure of the pressure relief is reached.

In a sixth aspect, the present invention is an apparatus for the removal of a particle from a fluid, the apparatus comprising:
a first endcap engageable with the inlet end of a hollow fibre filter, the first endcap including a first passage having an inlet engageable with a fluid input conduit; and an outlet into the filter; and
a second endcap engageable with the outlet end of the hollow fibre filter, the second endcap including a second passage having an outlet engageable with a fluid return conduit, and an inlet from the filter;
   the first passage and the second passage being independently sized such that in conjunction with a flow restriction means which restricts a flow of the fluid through the second passage, a predetermined exit liquid flow rate from at least one permeate outlet of the filter is achieved;
   at least a portion of the particulate is removed from the fluid exiting the at least one permeate outlet; and
   the maximum working pressure of the filter is not exceeded.

In an embodiment of the sixth aspect, the hollow fibre filter is preferably a haemodialysis filter.

In another embodiment of the sixth aspect, the first endcap further comprises a pressure relief valve, wherein the pressure relief valve provides a fluid pathway from the inlet end of the filter to the liquid sample supply when the liquid pressure applied to the filter approaches the maximum working pressure of the filter such that the maximum working pressure of the filter is not exceeded.

In a further embodiment of the second aspect, the microorganism is selected from the group of bacteria, protozoa or viruses. Preferably, the protozoa is selected from the group including Cryptosporidium and Giardia. Preferably, the virus is selected from those of the group including enterovirus, hepatitis A, rotavirus, noroviruses, astrovirus, reovirus, adenovirus and bacteriophage.

In a seventh aspect, the present invention is a method for the removal of a particulate from a fluid, the method comprising the steps of:
(i) capturing the particluate by passing the fluid through a hollow fibre filter, the hollow fibre filter having a first endcap engaged with the inlet end of a hollow fibre filter and a second endcap engageable with the outlet end of the hollow fibre filter, the first endcap including a first passage having an inlet engageable with a liquid input conduit; and an outlet into the filter so as to provide a fluid pathway between the filter and a liquid input conduit, and the second endcap including a second passage having an outlet engageable with a liquid return conduit, and an inlet from the filter so as to provide a fluid pathway between the filter and a liquid return conduit;
   wherein the first passage and the second passage are sized such that upon restriction of liquid flow rate through the liquid return conduit, a predetermined exit liquid flow rate from at least one permeate outlet of the filter is achieved;
   at least a portion of the particulate is removed from the fluid exiting the at least one permeate outlet; and
   the working pressure of the liquid passed through the hollow fibre filter is less than the maximum workable pressure of the filter.

In an embodiment of the seventh aspect, the hollow fibre filter is preferably a haemodialysis filter.

In another embodiment of the seventh aspect, the first endcap further comprises a pressure relief valve, wherein the pressure relief valve provides a fluid pathway from the inlet end of the filter to the liquid sample supply when the liquid pressure applied to the filter approaches the maximum working pressure of the filter such that the maximum working pressure of the filter is not exceeded.

In a further embodiment of the second aspect, the microorganism is selected from the group of bacteria, protozoa or viruses. Preferably, the protozoa is selected from the group including Cryptosporidium and Giardia. Preferably, the virus is selected from those of the group including enterovirus, hepatitis A, rotavirus, noroviruses, astrovirus, reovirus, adenovirus and bacteriophage.

### BRIEF DESCRIPTION OF DRAWINGS

The invention now will be described, by way of example only, and with reference to the accompanying drawings in which:

FIG. 1(a) shows a perspective view of an endcap of the present invention;

FIG. 1(b) shows a perspective view of the endcap of FIG. 1(a) in an alternate orientation;

FIG. 2 shows an example of the endcap of the present invention in combination with a pressure relief valve;

FIG. 3 shows a sectional view of the endcap of the present invention engaged with a hollow fibre filter and fluid inlet and outlet conduits; and

FIG. 4 shows an example of the endcaps of the present invention arranged in a configuration for the capture of a virus from a fluid.

### DETAILED DESCRIPTION OF THE INVENTION

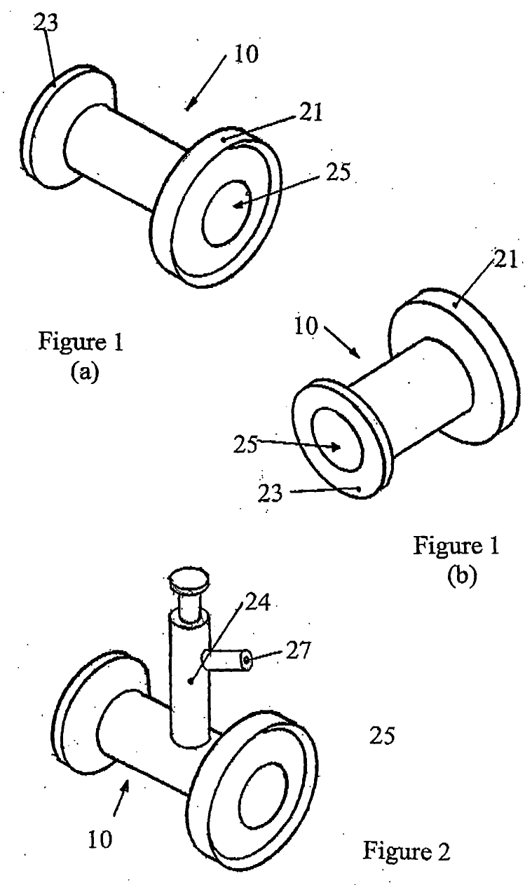

FIG. 1 shows an endcap 10 having a first end 21 engageable with the inlet end of a hollow fibre filter and a second end engageable with a liquid input conduit, and having a first passage 25 so as to provide a fluid pathway between the filter and the liquid conduit. The first passage 25 is circular in shape and has a diameter of about 21 millimetres. The endcap 10 is formed from a sterilisable material including metallic materials, in particular stainless steel, which is suitable for sterilisation.

FIG. 2 shows the endcap 10 of FIG. 1(a) and FIG. 1(b) having a pressure relief valve 24 in combination with the endcap 10 such that when a predetermined pressure within the endcap 10 is reached, the pressure relief valve 24 opens so as to provide a fluid pathway between the passage 25 and a release port 27. The pressure relief valve 24 is formed from a sterilisable material, in particular a stainless steel material. The pressure relief valve is adjustable for pressure release up to pressures of at least about 25 psi.

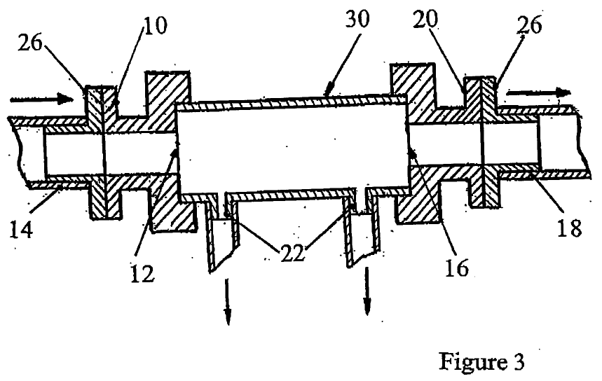

FIG. 3 shows the endcap of FIG. 1 when engaged with a hollow fibre filter. A first endcap 10 is engaged with the inlet end 12 of a hollow fibre filter 30 and engaged with an inlet conduit 14 via a first adaptor member 26(a), and a second endcap 20 engaged with the outlet end 16 of the hollow fibre filter 30 and engaged with a fluid return conduit 18 via a second adaptor member 26(b). A fluid pathway is formed from the inlet conduit 14 through the first adaptor member 26(a), through the first endcap 10, through the hollow fibre filter 30, through the second endcap 20, through the second adaptor member 26(a) and through the outlet conduit 18, as depicted by the arrows as shown. Permeate outlets 22 provide an outlet for the permeate (filtered liquid), as depicted by the arrows as shown.

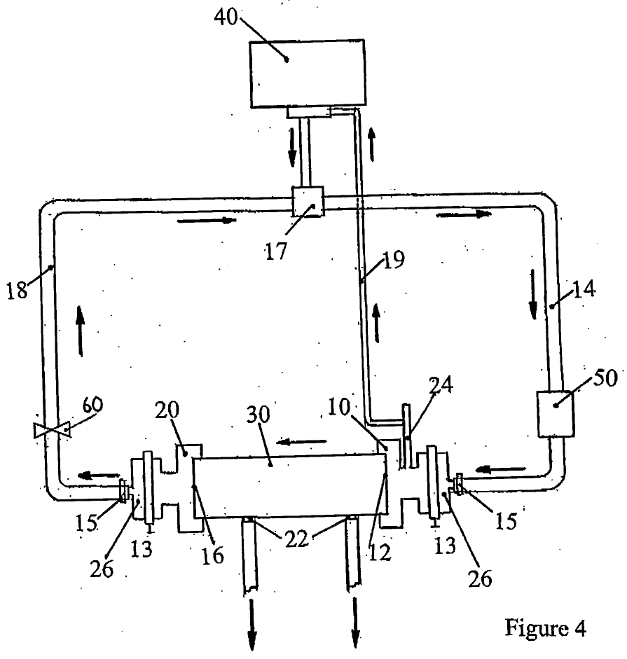

FIG. 4 shows an example of the endcaps of FIGS. 1-3 engaged with a hollow fibre filter 30 when used to isolate a virus from a finite liquid sample supply. The passages through the first endcap 10 and the second endcap 20 are sized such that upon restriction of fluid flow rate through the liquid return conduit 18 by a flow restriction means 60, a predetermined exit flow rate from the permeate outlets 22 of the filter is achieved such that the virus is captured within the hollow fibre filter 30, and the maximum working pressure of the filter is not exceeded. The flow restriction means 60 is selected from a group comprising a clamp means for compressing the liquid exit conduit, a flow restriction valve, a throttle valve and the inherent size of the second passage. The liquid input conduit 14, the first endcap 10, the hollow fibre filter 30, the second endcap 20 and the liquid return conduit 18 are connected in a loop arrangement, and the finite liquid sample supply is introduced into the loop arrangement between the liquid input conduit 14 and the liquid return conduit 18.

The liquid input conduit 14, the liquid return conduit 18 and a feed conduit 42 from the finite liquid sample supply 40 are fluidly connected by a T-junction member 17. A filtration mesh 44 is arranged between the finite liquid sample supply 40 and the closed loop arrangement. A first adaptor member 26(a) is provided between the first endcap 10 and the inlet conduit 14, and is affixed to the first endcap 10 by a clamp means 13, and is affixed to the fluid inlet conduit by a hose clamp 15. A second adaptor member 26(b) is engaged with the second endcap 20 via a clamp means 13 and is engaged with the fluid return conduit 18 via a hose clamp 15. A pressure relief valve provides a fluid pathway from the inlet end of the filter 20 to the liquid sample supply 40 when the liquid pressure applied to the hollow fibre filter 30 approaches the maximum working pressure of the filter such that the maximum working pressure of the filter is not exceeded. A Peristaltic Pump 50 is included so as to pump fluid through the closed loop arrangement.

### EXAMPLE

In the present example, the arrangement as shown in FIG. 4 is used for isolating and detecting a virus from a finite liquid sample supply, wherein the virus includes those of the group of enterovirus, Hepatitis A, rotavirus, Noroviruses astrovirus, reovirus, and adenovirus.

Example Parameters and Overview

The above method and apparatus is used for the detection and concentration of a virus from a large volume of water sample, for example of about 10-1000 litres using ultra filtration. The water samples are processed using a Peristaltic pumping system and a Hemoflow HF80S Hemodialysis disposable hollow fibre filtration cartridge (Fresenius Medical Care AG). The method used is a NATA accredited method for water, in the field of microbiological testing under the classes of test “8.70 waters including effluents”, 0.51 (potable waters), 0.52 (industrial waters), 0.55 (swimming pools and spas) and 0.56 (environmental waters). The three stage method for viral detection and environmental samples as used is shown below in chart 1. 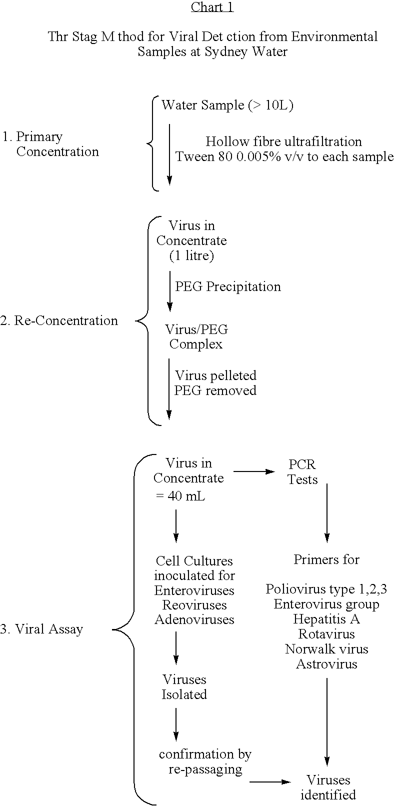
Source: Water Microbiology for the 21^{st} Century. Macquarie University Centre for Analytical Biotechnology & Sydney Water Corporation.
Apparatus

The following are preferred components used in the present example and system as shown in FIG. 4:

Peristaltic pump=Masterflex Peristaltic pump unit

Masterflex EasyLoad® pumphead liquid input conduit, inlet return conduit, feed conduit and pressure release valve conduit

Masterflex tubing (Pharmed I/P 82 Catalogue No 06485-82, silicon peroxide I/P 82 Catalogue No 96400-82.

First endcap and second endcap having an inner passage diameter of about 21 millimeters.

Hemoflow HF80S® cartridges Fresenius Medical Care Catalogue No CE0123

Filtration mesh—stainless steel mesh (13 mm diameter, 100 μm pore size eg. 150/45SS Metal Mesh Pty Ltd)

Container—sterile 1L centrifuge bottle (eg polycarbonate)

Finite liquid sample supply—contained in 10L FranRica™ water bag (Catalogue No EB-569)

Provision of venturi feed for water supply—FranRica™ water bag stand

Syringe—single use sterilised 60 mil syringe (Terumo Medical Corp)

Pressure relief valve engaged with first endcap

Parameters

The predetermined exit flow rate from the permeate outlets is in the range of from about 0.5 l/min to about 1.5l/min. The maximum working pressure of the hollow fibre filter is about 25 psi, and the pressure relief valve is adjusted to a pressure threshhold of about 20 psi such that the maximum pressure of the filter is not exceeded.

The first passage and the second passage of the first and second endcaps are circular and have a diameter of about 21 millimetres. The total volume of the loop arrangement and the feed conduit is about 340 ml. The finite liquid sample supply is contained within a FranRica™ waterbag, and has a supply volume of about 10 litres. The filtration mesh has a pore size of about 100 μm and a diameter of about 13 millimetres.

The finite liquid sample supply is introduced into the closed loop arrangement by venturi effect. Prior to commencing filtering, the finite liquid sample supply has a non-ionic surface active agent introduced, wherein the non-ionic surface active agent is Tween-80 in a concentration of about 0.005% v/v with the finite liquid sample supply. The carbonate buffer solution has a pH value of about 9.6.

Methodology

The Peristaltic pump is operated at a low pressure such that any air contained within the closed loop arrangement is purged from the closed loop arrangement by opening the pressure release valve located at the inlet site of the filter, and the air is returned to the FranRica™ waterbag by means of a conduit.

The liquid flow rate through the liquid return conduit is restricted by clamping the liquid return conduit by a clamping means, and the pressure applied to the inlet side of the filter by the Peristaltic pump is increased until the predetermined exit flow rate from the permeate outlets is obtained.

The step of capturing and concentrating the virus further comprises passing a buffer solution through the closed loop arrangement when about 50 ml to about 100 ml of residual liquid remains in the FranRica™ waterbag.

The liquid input conduit, the liquid return conduit and feed conduit are then disassociated, and the remaining liquid in the finite liquid sample supply and the feed conduit are transferred to a container having a first predetermined volume of buffer solution of about 600 ml.

The buffer solution is then drawn from the container by the peristaltic pump operated at a relatively low pressure, through the liquid inlet conduit, through the hollow fibre filter, passed through the liquid return conduit removed from the closed loop arrangement and returned to the container, until a second predetermined volume of about 300 ml of buffer solution remains in the container. The remaining liquid in the liquid inlet conduit, the hollow fibre filter and the liquid return conduit is then transferred to the container.

Nitrogen gas is used to purge any remaining liquid from the closed loop arrangement, and the filtration mesh is transferred to the container, such that substantially all of any virus initially present in the finite volume liquid supply is captured within the hollow fibre filter, or is present in the container containing the buffered solution and the mesh filter.

The step of removing the captured virus from the hollow fibre filter comprises:
(i) disengaging the first endcap and the second endcap from the hollow fibre filter;
(ii) closing the permeate outlets of the filter; and
(iii) backwashing the hollow fibre filter with a third predetermined volume of buffer solution such that the third volume of buffer solution is transferred to the container, and such that the virus from the finite liquid sample supply is now concentrated in the buffer solution in the container. The third predetermined volume of buffer solution is about 200 mls, and the hollow fibre filter is backwashed using a syringe. The 60 ml syringe maybe used such that the filter is flushed with about 3 volumes of buffer solution. The virus present in the buffer solution in the container is then further concentrated using the precipitation technique using polyethylene glycol-600 (PEG-6000) concentration techniques. The type of virus and quantity of virus can then be determined using cell culture and molecular biology techniques for predetermined viruses.
   Decontamination

The components of the above filter apparatus (with the exception of the filter cartridge), including conduits, clamps, couplings and pressure relief valve system may be reused between samples by performing the following:

In the case of positive controls, throughout each stage of disassembly, each component is placed in a storage container having 125 ppm sodium hypochlorite (1 mil of 12.5% v/w NaOCl in tap water);

For apparatus used to concentrate environmental samples, cleaning all fittings and tubing in warm (20° C.-50° C.) Tergazyme™ 1.0% w/v solution for at least 2 hours;
flushing with cold tap water; and
autoclaving components at 121° C. for 20 minutes.

Throughout the above method, it must be noted that aseptic techniques and good laboratory practice (glp) must be used at all times for occupational health and safety considerations, and so as to ensure that liquid from the finite liquid sample supply is not lost during the filtration process, so that as much virus contained in the liquid is captured in the filter as possible and observation of safety requirements when using the sodium hyperchlorite solution.

Furthermore, when a positive control test is run, all the virology safety aspects must be observed, in particular observance to the danger of creating aerosols potentially containing the virus.

Quality Control

A seeded positive control sample may be performed once per week for example to assess the ability of the operator to perform the procedure with respect to the acceptance criteria for the process. Furthermore, a second positive control can be used to gain recovery information from the finite liquid sample supply which is analysed using the above method and apparatus.

The acceptance criteria for quality control is ≦1.0 log loss of virus (positive control) over the entire process (ie. following ultra filtration and PEG precipitation). A suitable means of quality control is the use of a 10 litre sample of tap water, which is seeded with a high concentration of live attenuated poliovirus. Shown below is comparative data of virus detection and concentration using the above method and apparatus, in comparison with the use of standard techniques including the use of an Amicon® Micro Filtration by Millipore Pty Limited.

The present method and apparatus has a loss of virus well within the above acceptance criteria, and has means and variances with greater tolerances than that of the Amicon® method and apparatus. Furthermore, the above method in combination with the Hemoflow cartridge allows 10 litres of tap water to be filtered for a virus in about 10 minutes and when using the Amicon® filter, the time taken to filter 10 litres of water is about the same. In addition, the use of the Hemodialysis hollow fibre filters in the above method and apparatus are relatively inexpensive such that they may be used in a single use application and do not require to be cleaned after use, and calibrated and integrity examined by use of a positive control prior to use.

After use, the Hemodialysis filters, Hemoflow HF80S may be examined for integrity by attaching the manufacturers couplings and endcaps to the filter cartridge whilst the cartridge is full of permeate solution (for example water) and using a 60 mil syringe, pressurising the cartridge and examining the cartridge for evidence of bubbles emanating from cartridge fibres.

A comparison of Log virus loss between using the Hemoflow filter apparatus as described in the present invention and when using an Amicon® filter apparatus is shown below in Graph 1:

[image]

Comparative statistical data between the sample groups as illustrated in Graph 1 is shown Table 1. The Hemoflow filter apparatus provided a lower loss of virus than the Amicon® filter apparatus, and also had a lower variance as when the Amicon® was used. A confidence level of p<0.05 was used in the following statistical analysis:

TABLE 1

| | Hemoflow | Amicon |
|---|---|---|
| Mean | 0.345769 | 1.246341 |
| Variance | 0.033777 | 0.223464 |
| Observations | 26 | 41 |
| P(T <= t) one-tail | 8.67E−14 | |
| t Critical one-tail | 1.668636 | |
| P(T <= t) two-tail | 1.73E−13 | |
| t Critical two-tail | 1.997137 | |

Virus Concentration Using Polyethylene Glycol-600 (PEG-6000)

Precipitation of viruses with polyethylene glycol-6000 (PEG-6000) is an effective concentration method with slow precipitation of viruses in cold, high-salt conditions protecting viruses from chemical and physical denaturation. PEG-6000 is widely used to concentrate viruses from sample volumes of up to 1 litre. For clear water samples (drinking water, reuse water effluent), volumes greater than 1L are usually processed by ultrafiltration, followed by PEG-6000 processing as outlined in the Sydney Water methods. For water samples such as raw and primary sewage, 1L samples are processed by PEG-6000 precipitation alone, as outlined in this procedure. A positive control sample seeded with virus should be run with each batch of samples.

This technique is a NATA accredited method for water. Accreditation is in the field of Microbiological testing under classes of test ‘8.70 Waters including effluents’, 0.51 (potable waters), 0.52 (industrial waters), 0.53 (sewage), 0.54 (trade wastes) 0.55 (swimming pools and spas) and 0.56 (environmental waters).

The technique used in the present example is as follows:
(i) an appropriate amount of PEG-6000 is added to the centrifuge container containing the concentrated virus and buffer to give a final concentration of 8% w/v;
(ii) an appropriate volume of Tween-80 is added to the centrifuge container to give a final concentration of 1% v/v;
(iii) an appropriate volume of 1M CaCl₂ is added to the centrifuge container a final concentration of 0.5% v/v (eg add 5 mL of 1MCaCl₂ per litre of sample);
(iv) a sterile (autoclaved) magnetic flea is placed in the container, the container is then placed on a magnetic stirrer at approximately 4° C. and stirred for a minimum of 2 hours to a maximum of 24 h;
(v) the concentrated sample is then centrifuged for 1 hour at 7250 g at 4° C.;

(vi) immediately following centrifugation, the supernatant is discarded by vacuum the pellet resuspended in approximately 10 mL of sterile Phosphate Buffered Saline (PBS);
(vii) the pellet is vortex-mixed to resuspend the pellet, and the resuspended pellet is transferred to a sterile 50 mL centrifuge tube;
(viii) the centrifuge container is rinsed with approximately 10 mL MEM, and depending on the sample type, approximately 20 mL MEM alone can be used to resuspend the pellet and rinse the centrifuge bottle, rather than PBS/MEM. All rinses are pooled in the same 50 mL centrifuge container;
(ix) prior to sonication, the sample is vortex mixed for approximately 30 seconds;
(x) the sample is then sonicated in a sonication bath for 60 seconds (parameter settings: high power setting and low degas);
(xi) the sonicated sample is shaken vigorously for 15 minutes using a wrist action shaker on maximum speed at 800 oscillations per mninute;
(xii) immediately after shaking, the sample is centrifuged at 7250 g for 30 minutes at 4° C.;
(xiii) while the sample is centrifuging, 0.5 mL of Amphotericin-B (250 μg/mL-Fungizone) and 2.0 mL of Penicillin-Streptomycin (5000 μg/mL each) are added to appropriately labelled sterile 50 mL centrifuge tubes;
(xiv) the supernatant is decanted into the respective centrifuge tube.
(xv) the volume is made up to 40 mL with MEM. The colour of the indicator in MEM should remain red when added to the sample. If this is not the case, adjust the pH to 7 (using indicator strips to measure pH) by adding a drop at a time of 1M HCl or 1M NaOH as appropriate with a pasteur pipette. The sample should have a red colour. If pink then the sample is too alkaline. If yellow, then the sample is too acidic; and
(xvi) the processed sample is stored at 4° C. until inoculation. For long term storage, samples can be kept at approximately −80° C.
   Testing of Pressure Relief Valve

The pressure relief valve as used in the present example can be checked for activation pressure of 20 psi as used in this example by performing the steps of:
(i) engaging the endcap to which the relief valve is attached to a hydraulic hose, wherein the hose is engaged with a hydrostatic pump, the hydrostatic pump including a pressure gauge;
(ii) filling the assembly with water or other appropriate fluid;
(iii) bleeding air from the system using the pressure relief valve;
(iv) increasing the pressure from the hydrostatic pump until a pressure of 20 psi is reached.

At this stage, the pressure relief valve should be activated and water should trickle from the relief valve at about one drop per second, and upon the pressure being increased to 25 psi, water should fully flow from the pressure relief valve.

The opening pressure of the pressure relief valve should be adjusted so the above relief characteristics not be met.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An apparatus for isolating a microorganism from a liquid, the apparatus comprising:
a first endcap engageable with an inlet end of a hollow fibre filter, the first endcap including a first passage having an inlet engageable with a liquid input conduit; and an outlet into the filter; and
a second endcap engageable with an outlet end of the hollow fibre filter, the second endcap including a second passage having an outlet engageable with a liquid return conduit, and an inlet from the filter;
the first passage and the second passage being independently sized such that in conjunction with a flow restriction means which restricts a flow of the liquid through the second passage, a predetermined exit liquid flow rate from at least one permeate outlet of the filter is achieved;
the microorganism is captured within the hollow fibre filter; and
the maximum working pressure of the hollow fibre filter is not exceeded.

2. The apparatus according to claim 1, wherein the hollow fibre filter is a haemodialysis filter.

3. The apparatus according to claim 1, wherein the liquid is a finite liquid sample supply and the first endcap further comprises a pressure relief valve, wherein the pressure relief valve provides a fluid pathway from the inlet end of the filter to the finite liquid sample supply when the liquid pressure applied to the filter approaches the maximum working pressure of the filter such that the maximum working pressure of the filter is not exceeded.

4. The apparatus according to claim 1, wherein the predetermined exit liquid flow rate is in the range of from about 0.25 l/min to about 3.0 l/min.

5. The apparatus according to claim 1, wherein the predetermined exit liquid flow rate is about 1.5 l/min.

6. The apparatus according to claim 1, wherein the maximum working pressure of the hollow fibre filter is about 25 psi.

7. The apparatus according to claim 1, wherein the first passage and the second passage are circular, each having a diameter in the range from about 6 mm to about 30 mm.

8. The apparatus according to claim 1, wherein the first passage and the second passage each have a diameter of about 21 mm.

9. The apparatus according to claim 1, wherein the microorganism is selected from the group consisting of bacteria, protozoa and viruses.

10. A method for isolating a microorganism from a liquid, the method comprising the step of:
(i) capturing and concentrating the microorganism on a hollow fibre filter by passing a sample of the liquid through the hollow fibre filter, the hollow fibre filter having a first endcap engaged with the inlet end of a hollow fibre filter and a second endcap engageable with the outlet end of the hollow fibre filter, the first endcap including a first passage having an inlet engageable with a liquid input conduit; and an outlet into the filter so as to provide a fluid pathway between the filter and a liquid input conduit, and the second endcap including a second passage having an outlet engageable with a liquid return conduit, and an inlet from the filter so as to provide a fluid pathway between the filter and a liquid return conduit;
wherein the size of first passage and the second passage have been predetermined such that upon restriction of liquid flow rate through the liquid return conduit, a predetermined exit liquid flow rate from at least one permeate outlet of the hollow fibre filter is achieved; and
the pressure of the liquid passed through the hollow fibre filter is less than the maximum working pressure of the hollow fibre filter.

11. The method according to claim 10, further comprising the step of removing the captured microorganism from the hollow fibre filter, and optionally further concentrating the captured microorganism.

12. The method according to claim 10 wherein the hollow fibre filter is a haemodialysis filter.

13. The method according to claim 10 wherein the predetermined exit liquid flow rate is in the range of from about 0.25 l/min to about 3.0/min.

14. The method according to claim 10, predetermined exit liquid flow rate is about 1.5 l/min.

15. The method according to claim 10, wherein the maximum working pressure of the hollow fibre filter is about 25 psi.

16. The method according to claim 10, wherein the first passage and the second passage are circular, each having a diameter in the range from about 6 mm to about 30 mm.

17. The method according to claim 10, wherein the first passage and the second passage each have a diameter of about 21 mm.

18. The method according to claim 10, wherein the microorganism is selected from the group consisting of bacteria, protozoa and viruses.

19. An endcap for engagement with a hollow fibre filter, the endcap comprising:
a first end engageable with an end of the hollow fibre filter;
a passage having an inlet engageable with a liquid input conduit; and
an outlet into the filter;
wherein the passage has a diameter in the range of from about 6 mm to about 30 mm.

20. The endcap according to claim 19, wherein the hollow fibre filter is a haemodialysis filter.

21. The endcap according to claim 19 wherein the passage has a diameter of about 21 mm.

22. The endcap according to claim 19, further comprising a pressure relief valve, wherein the pressure relief valve provides a fluid pathway from the passage to atmosphere.
